(19) United States
(12) Patent Application Publication
(10) Pub. No.: US 2011/0039890 A1
(43) Pub. Date: Feb. 17, 2011

(54) 4-[2,3-Difluoro-6-(2-fluoro-4-methyl-phenylsulfanyl)-phenyl]-piperidine

(75) Inventors: Benny Bang-Andersen, Copenhagen (DK); Morten Jorgensen, Bagsvaerd (DK); Andre Faldt, Valby (DK); Neil Anderson, Frederiksberg (DK); Tine Bryan Stensbol, Vaerlose (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. LUNDBECK A/S, Valby-Copenhagen (DK)

(21) Appl. No.: 12/747,628

(22) PCT Filed: Dec. 11, 2008
(86) PCT No.: PCT/DK2008/050301

§ 371 Date: Oct. 21, 2010

Related U.S. Application Data
(60) Provisional application No. 61/013,892, filed on Dec. 14, 2007.

(30) Foreign Application Priority Data
Dec. 14, 2007 (DK) PA 200701792

(51) Int. Cl. A61K 31/451; C07D 211/20; A61P 25/00; A61P 25/24; A61P 25/28; A61P 25/16; A61P 25/22

(52) U.S. Cl. 514/317; 546/236

(57) ABSTRACT

The compound 4-[2,3-Difluoro-6-(2-fluoro-4-methyl-phenylsulfanyl)-phenyl]-piperidine according to the structure (formula I), and pharmaceutically acceptable salts thereof are provided for the treatment of CNS related disorders, such as: depressive disorder, dysthymic disorder; mood disorder due to a general medical condition; atypical depression; seasonal affective disorder; melancholia; treatment resistant depression; partial responders; depression associated with bipolar disorder, pain, Alzheimer's disease, psychosis, Parkinson's disease, Lewy body disease, Huntington's disease, multiple sclerosis or anxiety; general anxiety disorder, social anxiety disorder, panic attacks; phobia; social phobia, obsessive compulsive disorder; post traumatic stress disorder, acute stress; ADHD; and pain.

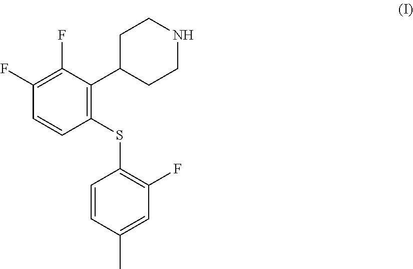

### FIELD OF THE INVENTION

The present invention relates to the compound 4-[2,3-difluoro-6-(2-fluoro-4-methyl-phenylsulfanyl)-phenyl]-piperidine, pharmaceutical compositions comprising said compound and therapeutic uses of said compound.

### BACKGROUND

Pain, and in particular chronic pain and depression are often co-morbid diseases, wherefore the provision of compounds which are effective on both diseases would be beneficial to the patient.

Selective serotonin (5-HT) reuptake inhibitors (SSRI) have for years been favoured by physicians for the treatment of many CNS diseases, such as depression and anxiety because they are effective and have a safety profile which is favourable compared to the previous generation of CNS drugs, i.e. the so-called tricyclics. Nevertheless, SSRIs are hampered by a significant fraction of non-responders, i.e. patients who do not or who do not fully respond to the treatment. Moreover, typically an SSRI does not begin to show an effect until after weeks of treatment. Finally, although SSRIs typically give rise to less adverse effects than tricyclics, the administration of SSRIs often brings about adverse effects, such as e.g. sleep disruption.

It is known that a combination of inhibition of the serotonin transporter (SERT) and an activity on one or more 5-HT receptors may result in a larger increase in the 5-HT level as compared to SSRIs and that this has been linked to faster onset of action and increase in efficacy as compared to SSRIs. It has for example been reported that the combination of pindolol, which is a 5-HT_{1A} receptor partial agonist, with a serotonin reuptake inhibitor (SRI) gives rise to faster onset of effect [Psych. Res., 125, 81-86, 2004]. It has also been found that the combination of an SRI with a 5-HT_{2C} receptor antagonist or inverse agonist (compounds having a negative efficacy at the 5-HT_{2C} receptor) provides a considerable increase in the level of 5-HT in terminal areas as compared to the SRI alone, as measured in microdialysis experiments [WO 01/41701]. As the therapeutic effectiveness of SRIs is believed to be linked to the increase in the 5-HT level, a combination of these activities would imply a shorter time to therapeutic effect in the clinic and an augmentation or potentiation of the therapeutic effect of the SRI.

The perception of pain is more complicated than a direct transmission of signals from an injured part of the body to specific receptors in the brain, and wherein the pain perceived is proportional to the injury. Rather, damage to peripheral tissue and injury to nerves may cause alterations in the central neural structures involved in pain perception affecting subsequent pain sensitivity. This neuroplasticity may bring about a central sensitization in response to longer lasting noxious stimuli, which may manifest itself as e.g. chronic pain, i.e. that the perception of pain remains even after the noxious stimulus has stopped, or as hyperalgesia, i.e. an increased response to a stimulus, which is normally painful. One of the more mysterious and dramatic examples of this is the “phantom limb syndrome”, i.e. the persistence of pain that existed in a limb prior to its amputation. For a recent review of central neuroplasticity and pain see Melzack et al in Ann. N.Y. Acad. Sci., 933, 157-174, 2001.

The central component to chronic pain may explain why chronic pain, such as e.g. neuropathic pain often responds poorly to classical analgesics, such as non-steroid anti-inflammatory drugs (NSAIDS) and opioid analgesics. Tricyclic antidepressants (TCA), typified by amitryptyline, have become standard for the treatment of neuropathic pain, and the effect is believed to be mediated by the combined inhibitory effect on the SERT and the noradrenaline (NA) transporter (NAT) [Clin Ther., 26, 951-979, 2004]. More recently, the so-called dual action antidepressants having an inhibitory effect on both the 5-HT and the NA reuptake have been used clinically for the treatment of neuropathic pain [Human Psychopharm., 19, S21-S25, 2004]. Examples of dual acting antidepressants are venlafaxine and duloxetine, and this class of antidepressants is often referred to as SNRIs.

Data on the use of SSRIs for treatment neuropathic pain are scarce, but generally suggest a limited effect [Bas. Clin. Pharmacol., 96, 399-409, 2005]. In fact, it has been hypothesized that SSRIs are only weakly antinociceptive by themselves but that inhibition of the SERT augments the antinociceptive effect of NA reuptake inhibition. This notion is supported by a review of 22 animal and five human studies showing that SNRIs have superior antinociceptive effect compared to NA reuptake inhibitors, which again are superior to SSRIs [Pain Med. 4, 310-316, 2000].

Thus, it would seem that compounds inhibiting the SERT and the 5-HT_{2C} receptor and which also inhibits the noradrenaline transporter would provide compounds effective in the treatment of affective disorders and pain.

The international patent application published as WO 2003/029232 discloses e.g. the compound 4-[2-(4-methylphenylsulfanyl)phenyl]piperidine as a free base and the corresponding HCl salt. The compound is reported to be an inhibitor of the SERT and the 5-HT_{2C} receptor, and is said to be useful for the treatment of affective disorders, e.g. depression and anxiety. The international patent application published as WO 2004/087156 also discloses a range of phenylsulfanylphenyl piperidins with the same pharmacological profile as the compound disclosed in the '232 application.

### SUMMARY OF THE INVENTION

The present inventors have surprisingly found that compound I, i.e. 4-[2,3-difluoro-6-(2-fluoro-4-methyl-phenylsulfanyl)-phenyl]-piperidine and pharmaceutically acceptable acid addition salts thereof, are potent inhibitors of the SERT, inhibits the 5-HT_{2A} and 5-HT_{2C} receptors and inhibits the NA transporter (NAT). Thus, in one embodiment, the invention relates to 4-[2,3-difluoro-6-(2-fluoro-4-methyl-phenylsulfanyl)-phenyl]-piperidine and pharmaceutically acceptable acid addition salts thereof.

In one embodiment, the invention relates to a method of treatment comprising the administration of a therapeutically effective amount of compound I to a patient in need thereof.

In one embodiment, the invention relates to a pharmaceutical composition comprising compound I and at least one pharmaceutically acceptable carrier or diluent.

In one embodiment, the invention relates to compound I for use in therapy.

In one embodiment, the invention relates to compound I for use in the treatment of certain diseases.

In one embodiment, the invention relates to the use of compound I in the manufacture of a medicament for the treatment of certain diseases.

### DETAILED DESCRIPTION OF THE INVENTION

The structure of 4-[2,3-difluoro-6-(2-fluoro-4-methyl-phenylsulfanyl)-phenyl]-piperidine is

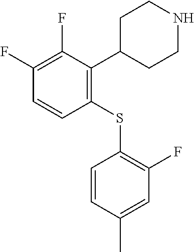

and the invention relates to compound I which is defined as this compound and pharmaceutically acceptable acid addition salts thereof.

In one embodiment, said acid addition are salts of acids that are non-toxic. Said salts include salts made from organic acids, such as maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, malonic, mandelic, cinnamic, citraconic, aspartic, stearic, palmitic, itaconic, glycolic, p-aminobenzoic, glutamic, benzenesulfonic, theophylline acetic acids, as well as the 8-halotheophyllines, for example 8-bromotheophylline. Said salts may also be made from inorganic salts, such as hydrochloric, hydrobromic, sulfuric, sulfamic, phosphoric and nitric acids.

Oral dosage forms, and in particular tablets and capsules, are often preferred by the patients and the medical practitioner due to the ease of administration and the consequently better compliance. For tablets and capsules, it is preferable that the active ingredients are crystalline. In one embodiment, compound I is crystalline.

Crystals used in the present invention may exist as solvates, i.e. crystals wherein solvent molecules form part of the crystal structure. The solvate may be formed from water, in which case the solvates are often referred to as hydrates. Alternatively, the solvates may be formed from other solvents, such as e.g. ethanol, acetone, or ethyl acetate. The exact amount of solvate often depends on the conditions. For instance, hydrates will typically loose water as the temperature is increased or as the relative humidity is decreased. Compounds, which do not change or which change only little when conditions, such as e.g. humidity change are generally regarded as better suited for pharmaceutical formulations.

Some compounds are hygroscopic, i.e. they absorb water when exposed to humidity. Hygroscopicity is generally regarded as an undesired property for compounds, which are to be presented in a pharmaceutical formulation, in particular in a dry formulation, such as tablets or capsules. In one embodiment, the invention provides crystals with low hygroscopicity.

For oral dosage forms using crystalline active ingredients it is also beneficial if said crystals are well-defined. In the present context, the term “well-defined” in particular means that the stoichiometry is well-defined, i.e. that the ratio between the ions forming the salt is the ratio between small integers, such as 1:1, 1:2, 2:1, 1:1:1, etc. In one embodiment, the compounds of the present invention are well-defined crystals.

The solubility of an active ingredient is also of significance for the choice of dosage form as it may have a direct impact on bio-availability. For oral dosage forms, a higher solubility of the active ingredient is generally believed to be beneficial as it increases the bio-availability.

The pharmacological profile of compound I is shown in the examples and may be summarised as follows. Compound I inhibits the reuptake of 5-HT and NA and it inhibits the 5-HT_{2A} and the 5-HT_{2C} receptors. Thus, compound I may be useful in the treatment of affective disorders, such as depression and anxiety, but its pharmacological profile may also make it useful in the treatment of additional indications.

5-HT_{2A} and 5-HT_{2C} receptors are located e.g. on NA and dopaminergic (DA) neurons, respectively, where activation exerts a tonic inhibitory influence on the NA and DA release, respectively, and 5-HT_{2A} and 5-HT_{2C} receptor antagonists will effect an increase in the NA and DA levels, respectively. On this background it may be hypothesized that 5-HT_{2A} and 5-HT_{2C} receptor antagonists are particular well-suited for the treatment of depression which is refractory to the treatment with SRIs (treatment resistant depression, TRD, or refractory depression) [Psychopharmacol. Bull., 39, 147-166, 2006].

A segment of depressed patients will respond to treatment with e.g. SSRI in the sense that they will improve on clinically relevant depression scales, such as MADRD (Montgomery Aasberg Depression Rating scale) and HAMD (Hamilton Depression Rating Scale), but where other symptoms, such as sleep disturbances and cognitive impairment remain. In the present context, these patients are referred to as partial responders. Due to the 5-HT_{2A} receptor and 5-HT_{2C} receptor antagonism of compound I, which is believed to be reflected in the effects on sleep, compound I may be useful in the treatment of partial responders, or rephrased that treatment of depressed patients with compounds of the present invention will reduce the fraction of partial responders.

Sleep disturbances seem to be a general adverse affect of most antidepressants. In particular, SSRI, NRI and SNRI are reported to give rise to problems with sleep initiation and maintenance and problems with insomnia are often reported, too [Int. Clin. Psychpharm., 21 (suppl 1), S25-S29, 2006]. Others report that such compounds give rise to suppressed REM sleep, increased sleep latency, less efficient sleep, increase in nocturnal awakenings, and fragmentation of sleep [Hum. Psychopharm. Clin. Exp., 20, 533-559, 2005].

It is generally speculated that the adverse sleep effects are caused by stimulation of the 5-HT_{2A} and the 5-HT_{2C} receptors. R. L. Fish reports in Bioorg. Med. Chem. Lett., 15, 3665-3669, 2005 that certain 4-fluorosulfonylpiperidines, which are highly selective 5-HT_{2A} receptor antagonists are effective in increasing the slow wave sleep duration and decreasing the number of awakenings in rats. These pre-clinical observations are confirmed by clinical findings. Ritanserin, a 5-HT_{2A} receptor antagonist, has been shown to increase the total sleep time, the slow wave sleep duration, the REM sleep duration, and improve the subjective sleep quality in humans [Clin. Neurophys. 113, 429-434, 2002]. Nefazodone, a potent inhibitor of 5-HT_{2A} receptors and a weak inhibitor of the 5-HT and the NA reuptake, has in clinical trials been shown to increase sleep continuity and total REM sleep time, and to reduce the number of awakenings [Biol. Psychiatry, 44, 3-14, 1998]. Similarly, trazodone, which is a 5-HT_{2A} receptor antagonist and a moderate inhibitor of the 5-HT reuptake, has been shown to improve the clinical scores HAS (sleep disorders) and HRSD (premature morning awakening, lack of sound sleep and initiating sleep) [Psychiatr. Clin. Neurosci., 53, 193-194, 1999]. Sharpley in Neuropharmacology, 33, 467-471, 1994 reports that 5-HT_{2A} and in particular 5-HT_{2C} receptor antagonists improve the slow-wave sleep.

The above findings and observations suggest that the identification of compounds having an inhibitory effect of the 5-HT and/or NA reuptake in combination with a 5-HT_{2A/C} receptor antagonistic activity would provide compounds suitable for the treatment of affective disorders, such as e.g. depression and anxiety, without or with reduced adverse sleep effects.

Bipolar disorder was formerly known as manic-depressive illness and it is characterised by recurrent episodes of mania and depression. A major challenge in the treatment of bipolar depression (or the depression associated with bipolar disease) is to avoid the manic shift, i.e. avoid that depressed patients develop manic episodes as a consequence of the anti-depressive treatment. Treatment-emergent mania has been reported for a significant fraction of patients with bipolar depression after treatment with anti-depressants [J. Clin. Psych., 67, suppl 11, 18-21, 2006]. Typically manic episodes are treated with antipsychotics, such as quetiapine or olanzapine, both of which exhibit 5-HT_{2A} receptor antagonistic effects or with lithium. A compound combining 5-HT and NA reuptake inhibition with antagonistic effect on the 5-HT_{2A} receptor would thus seem to be the ideal compound for the treatment of bipolar depression avoiding a manic shift.

Sleep disturbances and anxiety are hallmarks of post traumatic stress disorder (PTSD), therefore compounds having an effect on both these symptoms would be well-suited for the treatment of this disease.

Melancholia is a particular subtype of depression often connected to severe depression; this type of depression is also referred to as melancholic depression. Melancholia is associated with anxiety, dread of the future, insomnia, and loss of appetite. Compounds that inhibit both the 5-HT and the NA reuptake, such as e.g. venlafaxine, have been shown to be particular effective in the treatment of patients with severe depression and melancholia [Depres. Anxiety, 12, 50-54, 2000].

Attention deficit hyperactivity disorder (ADHD) is one of the most common neurobehavioral disorders. ADHD is characterised by the presence of a triad of social and communicative impairments with restricted, repetitive or stereotyped behaviours. ADHD usually starts in childhood or adolescence, but symptoms may continue into adulthood. Atomoxetine is currently the only nonstimulant approved by FDA for the treatment of ADHD [Drugs, 64, 205-222, 2004]. Atomoxetine is a NA reuptake inhibitor, and this suggests that compound I may be used in the treatment of ADHD. In addition, compounds that are antagonists of the 5-HT_{2A/C} receptor may have a sleep improving effect as discussed above, which is beneficial in the treatment of ADHD.

The pharmacological profile of compound I and in particular the combined facilitation of 5-HT and NA neurotransmission via inhibitory effect of the SERT and NAT and antagonism of 5-HT_{2A} and 5-HT_{2C} receptors suggests that compound I may be particularly useful in the treatment of pain and in particular chronic pain. Special mention is made of the use of compound I in the treatment of pain and in particular chronic pain in patients who are also suffering from an affective disorder, such as depression and anxiety.

As shown in the examples, compound I has, in fact, in animal tests been shown to have a marked and dose-dependent effect in the treatment of neuropathic pain.

In one embodiment, the invention relates to the treatment of a disease selected from major depressive disorder; dysthymic disorder; mood disorder due to a general medical condition; atypical depression; seasonal affective disorder; melancholia; treatment resistant depression; partial responders; depression associated with bipolar disorder, pain, Alzheimer's disease, psychosis, Parkinson's disease, Lewy body disease, Huntington's disease, multiple sclerosis or anxiety; general anxiety disorder; social anxiety disorder; panic attacks; phobia; social phobia, obsessive compulsive disorder; post traumatic stress disorder; acute stress; ADHD; and pain.

In one embodiment the invention relates to compound I for use in the treatment of a disease selected from major depressive disorder; dysthymic disorder; mood disorder due to a general medical condition; atypical depression; seasonal affective disorder; melancholia; treatment resistant depression; partial responders; depression associated with bipolar disorder, pain, Alzheimer's disease, psychosis, Parkinson's disease, Lewy body disease, Huntington's disease, multiple sclerosis or anxiety; general anxiety disorder; social anxiety disorder; panic attacks; phobia; social phobia, obsessive compulsive disorder; post traumatic stress disorder; acute stress; ADHD; and pain.

In one embodiment, the invention relates to the use of compound I in the manufacture of a medicament for the treatment of a disease selected from major depressive disorder; dysthymic disorder; mood disorder due to a general medical condition; atypical depression; seasonal affective disorder; melancholia; treatment resistant depression; partial responders; depression associated with bipolar disorder, pain, Alzheimer's disease, psychosis, Parkinson's disease, Lewy body disease, Huntington's disease, multiple sclerosis or anxiety; general anxiety disorder; social anxiety disorder; panic attacks; phobia; social phobia, obsessive compulsive disorder; post traumatic stress disorder; acute stress; ADHD; and pain.

In one embodiment, said pain is chronic pain which may further be selected from phantom limb pain, neuropathic pain, diabetic neuropathy, post-herpetic neuralgia (PHN), carpal tunnel syndrome (CTS), HIV neuropathy, complex regional pain syndrome (CPRS), trigeminal neuralgia/trigeminus neuralgia/tic douloureux, surgical intervention (e.g. post-operative analgesics), diabetic vasculopathy, capillary resistance or diabetic symptoms associated with insulitis, pain associated with angina, pain associated with menstruation, pain associated with cancer, dental pain, headache, migraine, tension-type headache, trigeminal neuralgia, temporomandibular joint syndrome, myofascial pain muscular injury, fibromyalgia syndrome, bone and joint pain (osteoarthritis), rheumatoid arthritis, rheumatoid arthritis and edema resulting from trauma associated with burns, sprains or fracture bone pain due to osteoarthritis, osteoporosis, bone metastases or unknown reasons, gout, fibrositis, myofascial pain, thoracic outlet syndromes, upper back pain or lower back pain (wherein the back pain results from systematic, regional, or primary spine disease (radiculopathy), pelvic pain, cardiac chest pain, non-cardiac chest pain, spinal cord injury (SCI)-associated pain, central post-stroke pain, cancer neuropathy, AIDS pain, sickle cell pain or geriatric pain.

In an embodiment, the compound of the invention is administered in an amount of about 0.001 to about 100 mg/kg body weight per day.

A typical oral dosage is in the range of from about 0.001 to about 100 mg/kg body weight per day, preferably from about 0.01 to about 50 mg/kg body weight per day, administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

A typical oral dosage for adults is in the range of 1-100 mg/day of a compound of the present invention, such as 1-30 mg/day, or 5-25 mg/day. This may typically be achieved by the administration of 0.1-50 mg, such as 1-25 mg, such as 1, 5, 10, 15, 20 or 25 mg of the compound of the present invention once or twice daily.

A “therapeutically effective amount” of a compound as used herein means an amount sufficient to cure, alleviate or partially arrest the clinical manifestations of a given disease and its complications in a therapeutic intervention comprising the administration of said compound. An amount adequate to accomplish this is defined as “therapeutically effective amount”. The term also includes amounts sufficient to cure, alleviate or partially arrest the clinical manifestations of a given disease and its complications in a treatment comprising the administration of said compound. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatment are two separate aspect of the invention. The patient to be treated is preferably a mammal, in particular a human being.

The compounds of the present invention may be administered alone as a pure compound or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, cremes, gels, inhalants, dermal patches, implants, etc.

Conveniently, the compounds of the invention are administered in a unit dosage form containing said compounds in an amount of about 0.1 to 50 mg, such as 1 mg, 5 mg 10 mg, 15 mg, 20 mg or 25 mg of a compound of the present invention.

For parenteral routes such as intravenous, intrathecal, intramuscular and similar administration, typically doses are in the order of about half the dose employed for oral administration.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colorings, flavourings, preservatives etc. may be used provided that they are compatible with the active ingredients.

Capsules comprising a compound of the present invention may be prepared by mixing a powder comprising said compound with microcrystalline cellulose and magnesium stearate and place said powder in a hard gelatine capsule. Optionally, said capsule may be coloured by means of a suitable pigment. Typically, capsules will comprise 0.25-20% of a compound of the present invention, such as 0.5-1.0%, 3.0-4.0%, 14.0-16.0% of a compound of the present invention. These strengths can be used to conveniently deliver 1, 5, 10, 15, 20 and 25 mg of a compound of the present invention in a unit dosage form.

Solutions for injections may be prepared by dissolving the active ingredient and possible additives in a part of the solvent for injection, preferably sterile water, adjusting the solution to the desired volume, sterilising the solution and filling it in suitable ampoules or vials. Any suitable additive conventionally used in the art may be added, such as tonicity agents, preservatives, antioxidants, etc.

Compound I may either be administered alone or in combination with another therapeutically active compound, wherein the two compounds may either be administered simultaneously or sequentially. Examples of therapeutically active compounds which may advantageously be combined with compound I include sedatives or hypnotics, such as benzodiazepines; anticonvulsants, such as lamotrigine, valproic acid, topiramate, gabapentin, carbamazepine; mood stabilizers such as lithium; dopaminergic drugs, such as dopamine agonists and L-Dopa; drugs to treat ADHD, such as atomoxetine; psychostimulants, such as modafinil, ketamine, methylphenidate and amphetamine; other antidepressants, such as mirtazapine, mianserin and buproprion; hormones, such as T3, estrogen, DHEA and testosterone; atypical antipsychotics, such as olanzapine and aripiprazole; typical antipsychotics, such as haloperidol; drugs to treat Alzheimer's diseases, such as cholinesterase inhibitors and memantine, folate; S-Adenosyl-Methionine; immunomodulators, such as interferons; opiates, such as buprenorphins; angiotensin II receptor 1 antagonists (AT1 antagonists); ACE inhibitors; statins; and alpha 1 adrenergic antagonist, such as prazosin.

Compound I, free base may be prepared e.g. as outlined in WO 2004/087156. Salts may be prepared by addition of an appropriate acid followed by precipitation. Precipitation may be brought about by e.g. cooling, removal of solvent, addition of another solvent or a mixture thereof. Alternatively, compound I may be prepared as disclosed in the examples.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### EXAMPLES

Unless otherwise indicated, LC/MS was run in the following set-up.

LC/MS, general: Solvent system: A=water/TFA (100:0.05) and B=water/acetonitrile/TFA (5:95:0.035) (TFA=trifluoroacetic acid). The retention time (RT) is expressed in minutes. MS instruments are from PESciex (API), equipped with APPI-source and operated in positive ion mode.

Method: API 150EX and Shimadzu LC8/SLC-10A LC system. Column: 30×4.6 mm Waters Symmetry C18 with 3.5 μM particles operated at room temperature. Linear Gradient elution with 10% B to 100% B in 4 min and a flow rate of 2 ml/min.

### Example 1

### Pharmacological Profile

IC₅₀ (nM) values Compound I inhibition of the reuptake in rat synaptosomes:

[³H]-serotonin: 2.4
[³H]-noradrenaline: 12

Affinity (Kᵢ, nM) for compound I to human serotonin receptors calculated from the Cheng-Prusoff equation

### 5-HT_{2A}: 13

### 5-HT_{2C}: 4.9

In a functional assay, compound I is shown to be an antagonist of the 5-HT_{2A} receptor with a K_{b} around 130 nM as measured in a FLIPR assay. Similarly, compound I is an antagonists of the 5-HT_{2C} receptor with a K_{b} around 35 nM.

### Example 2

### Synthesis of Compound I

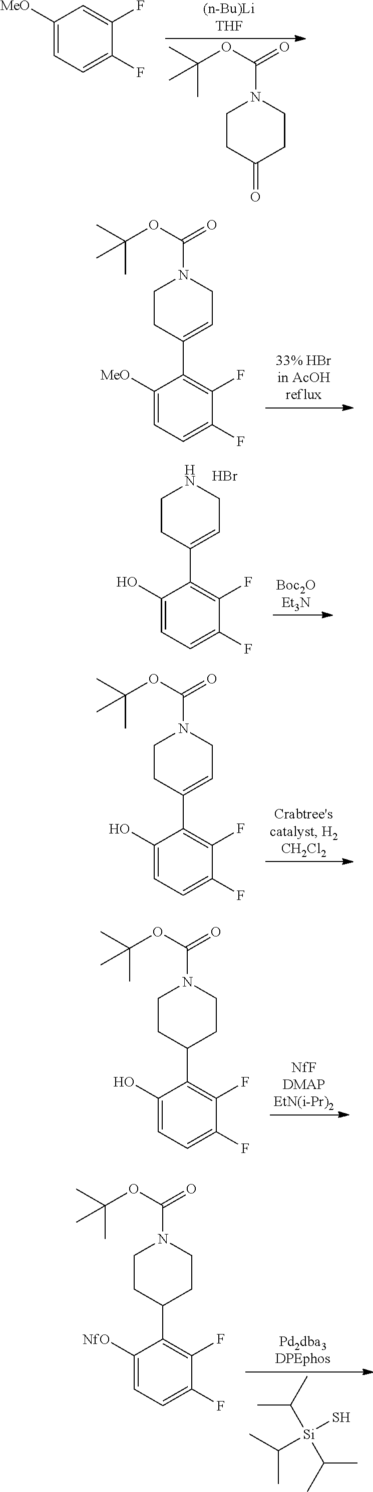

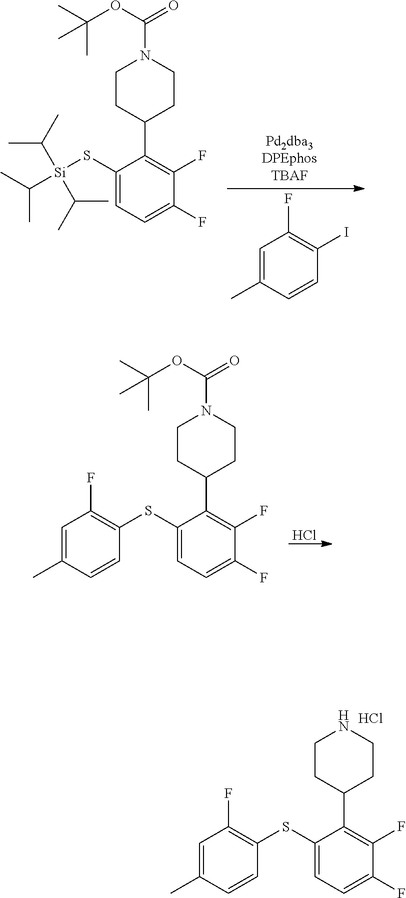

Step 1: 3,4-Difluoroanisol (25.0 g) was dissolved in tetrahydrofuran (200 mL), and the solution was cooled to −78° C. n-Butyl lithium (1.7 M in hexanes, 102 mL) was added over 1 h maintaining the temperature below −70° C. After 3 h at −78° C., 4-oxo-piperidine-1-carboxylic acid tert-butyl ester (31.2 g in 100 mL tetrahydrofuran) was added at such a rate that the temperature was maintained below −65° C. Next morning, the crude mixture was washed with saturated aqueous ammonium chloride (200 mL) and water (100 mL). The organic layer was dried over magnesium sulphate and concentrated in vacuo to afford the crude product. This material was purified by chromatography on silica gel (eluent: ethyl acetate/heptane 1:1) to afford the product (27.7 g; contaminated with 4-oxo-piperidine-1-carboxylic acid tert-butyl ester).

Step 2: The product from the previous step was refluxed in a mixture of 33% hydrogen bromide in acetic acid (50 mL) and 48% aqueous hydrogen bromide (50 mL) overnight. Next morning, the mixture was cooled to room temperature and the precipitated solid (12.7 g) was collected by filtration and used in the next step.

Step 3: A portion of the product from the previous step (7.7 g) was dissolved in ethanol (150 mL). Triethyl amine (3.8 mL) was added followed by di-tent-butyl dicarbonate (5.8 g) in small portions over 5 minutes. The mixture was allowed to stir over the weekend at room temperature (rt). The precipitated product was filtered off, and the filtrate was concentrated in vacuo to produce a second crude product fraction. This material was partitioned between diethyl ether (100 mL) and water (100 mL) and 10% aqueous sodium hydroxide (20 mL). The organic layer was washed with saturated aqueous sodium chloride (100 mL) and dried over magnesium sulphate. Filtration and concentration in vacuo afforded a second crop of the product (overall yield 8.03 g).

Step 4: A portion of the product from the previous step (3.0 g) was dissolved in methylene chloride (100 mL). (1,5-Cyclooctadiene)(pyridine)(tri-cyclo-hexyl-phosphane)iridium(I) hexafluorophosphate (Crabtree's catalyst; 775 mg; 10%) was added, and the mixture was treated with hydrogen gas (3 bar) using a Parr shaker Fresh catalyst was added several times over ˜24 h (totally 30%). Filtration yielded a white solid, which was used in the next step.

Step 5: The crude mixture from the previous step was dissolved in N-N-dimethyl formamide (20 mL). Ethyl-di-iso-propyl amine (Hünig's base; 0.76 g) and 4-dimethylamino-pyridine (0.12 g) were added followed by 1,1,2,2,3,3,4,4,4-nonafluoro-butane-1-sulfonyl fluoride (NfF; 1.62 g). After 1 h, the volatiles were removed in vacuo, and the crude product was purified by chromatography on silica gel (eluent: ethyl acetate/heptane 1:4) to produce the desired product (2.04 g).

Step 6: The product from the previous step (2.04 g) was added to a flask containing sodium tert-butoxide (0.45 g) and dry toluene (25 mL). The mixture was degassed with argon before it was added to a flask containing a degassed mixture of tris(dibenzylideneacetone)dipalladium(0) (Pd₂dba₃; 166 mg) and bis[(2-diphenyl-phosphanyl)phenyl]ether (DPEphos; 195 mg) in dry toluene (10 mL). Finally, tri-iso-propyl-silanethiol (0.78 mL) was added, and the mixture was stirred under argon at 100° C. overnight. After cooling to room temperature, the crude mixture was purified by chromatography on silica gel (eluent ethyl acetate/heptane 1:9) to give the desired product (181 mg).

Step 7: The product from the previous step was dissolved in dry toluene (8 mL) under argon. A portion of this stock solution (1 mL) was added to a reaction-vial in a Mettler-Toledo Bohdan block using an atmosphere of argon to exclude air. 2-Fluoro-1-iodo-4-methyl-benzene (0.33 mmol; prepared from 2-fluoro-4-methyl-phenylamine according to a general literature procedure [S. E. Tunney and J. K. Stille, J. Org. Chem., 52, 748-53 (1987)]) was added as a toluene solution (1 mL) followed by 0.5 mL of a freshly prepared toluene stock of solution of tris(dibenzylideneacetone)dipalladium(0) (Pd₂ dba₃) and bis[(2-diphenyl-phosphanyl)phenyl]ether DPEphos (corresponding to 0.3 equivalents palladium and 0.6 equivalents DPEphos). Potassium tert-butoxide (0.66 mmol) was added followed by tetra-n-butyl ammonium fluoride (TBAF; 1.0M in THF; 80 microliter). The mixture was stirred at 100° C. overnight under argon. Next morning, the volatiles were removed using a Genevac instrument. The residue was dissolved in methanol (4 mL) and loaded onto a VacMaster SCX-column (activated with 10% acetic acid in methanol). The product was eluted with acetonitrile. The volatiles were removed in vacuo. The residue was dissolved in methanol (1.5 mL) and 4M HCl in diethyl ether (1.5 mL) was added. The mixture was shaken at room temperature over the weekend before the volatiles were removed in vacuo. The residue was dissolved in dimethyl sulfoxide (0.18 mL) and filtered. A few drops of 20% acetonitrile in water were added, and the mixture was filtered again. The product was isolated by preparative LC/MS as described, concentrated in vacuo, and the product was dissolved in dimethyl sulfoxide (0.78 mL) to give a 10 mM solution. LC/MS-data: Method 14, retention time (UV) 2.152 min; UV-purity 79.5%; ELS-purity 100%; mass observed 337.407.

### Example 3

### Synthesis of Compound I

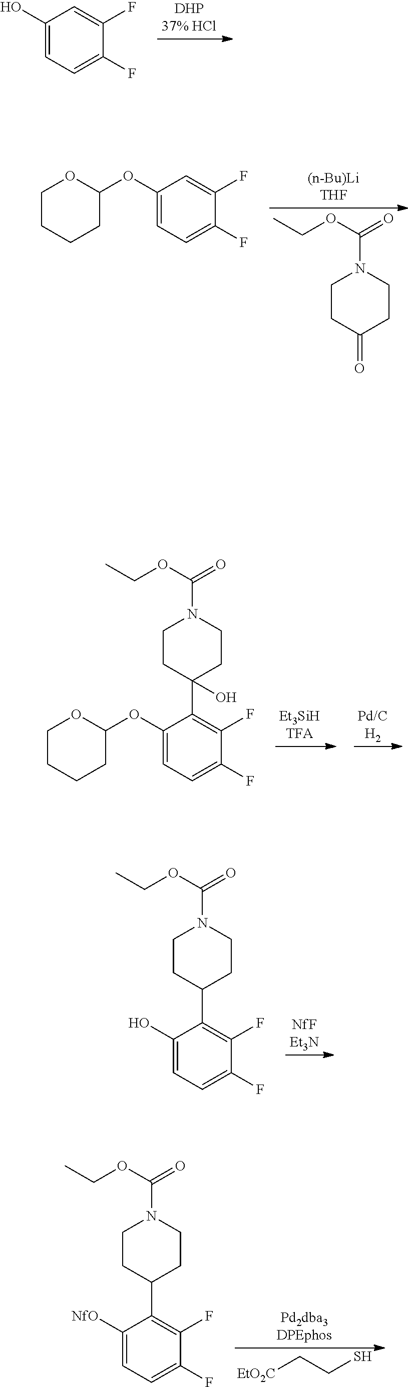

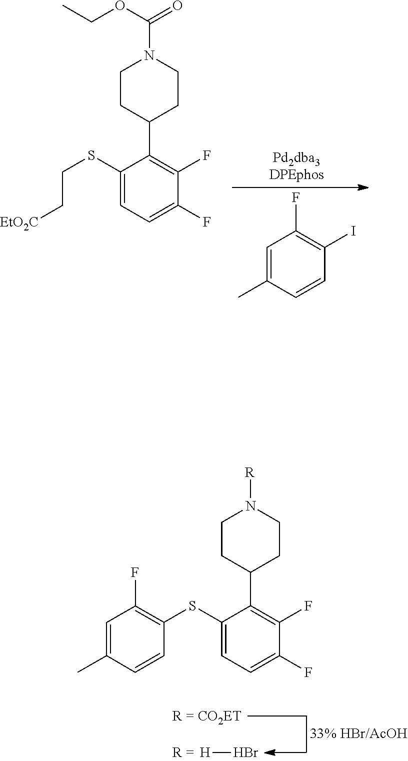

Step 1: 3,4-difluorophenol (100 g) was dissolved in 3,4-dihydro-2H-pyran (DHP; 280 mL). 0.5 mL concentrated aqueous hydrogen chloride was added, and the mixture was stirred overnight at room temperature. The crude mixture was extracted with saturated aqueous sodium hydrogen carbonate (200 mL) and diethyl ether (400 mL), and the organic layer was washed with saturated aqueous sodium chloride (200 mL) and dried over magnesium sulphate. Filtration and concentration in vacuo afforded the desired compound (169 g) as a pale yellow oil.

Step 2: A solution of the product from the previous step (a different batch; 214.2 g) in tetrahydrofuran (2 L) was purged with nitrogen and cooled to −35° C. A solution of n-butyl lithium (10M in hexanes; 120 mL) was added over 70 minutes, and the resulting mixture was stirred at −35° C. for 260 minutes. Then 4-oxy-piperidine-1-carboxylic acid ethyl ester (205.4 g) was added drop-wise over 70 minutes maintaining the temperature below −30° C., before the mixture was allowed to stir overnight at rt. Next morning the mixture was cooled to 0° C., and the 2M aqueous hydrogen chloride (200 mL) was added. The mixture was stirred at room temperature for 3 h. The crude mixture was partitioned between water (500 mL) and ethyl acetate (200 mL). The aqueous layer was extracted with ethyl acetate (200 mL). The combined organic layers were washed with 15% aqueous sodium chloride (3×200 mL), and co-concentration in vacuo with toluene (3×250 mL) to give a yellow oil (442.4 g).

Steps 3+4: The product from the previous step was added to triethyl silane (160 mL), and the mixture was heated to 60° C. Trifluoro acetic acid (TFA; 250 mL) was added followed by additional triethyl silane (50 mL). After 90 minutes, activated charcoal (25 g) was added, and the mixture was stirred at 70° C. for 0.5 h. Ethanol (500 mL) was added, and the mixture was stirred overnight at rt. Next morning, the mixture was heated to reflux for 1 h, before it was filtered while warm. The filtrate was concentrated in vacuo. The residue was stirred in ethanol (100 mL) at 0° C. for 2.5 h. The precipitated solid (7.7 g) was collected by filtration. The filtrate was stirred in ethyl aceteate (50 mL) and heptane (300 mL) to give a second portion of the product as a hard off-white material (153.8 g), which was isolated by filtration. The combined product fractions were dissolved in tetrahydrofuran/ethanol (1:3; 1.5 L) and treated with Pd/C (5.4 g) and hydrogen gas (3 bar) at room temperature using a Parr shaker. The catalyst was filtered off, and the filtrate was concentrated in vacuo to give a solid material, which was stirred in heptane (300 mL) and then isolated by filtration to give a white solid (144.6 g).

Step 5: A suspension of the product from the previous step (a different batch; 175 g) in acetonitrile (1.5 L) and triethyl amine (255 mL) was treated with 1,1,2,2,3,3,4,4,4-nonafluoro-butane-1-sulfonyl fluoride (NfF; 142.6 mL) at room temperature. After 25 min, the mixture was concentrated in vacuo to afford the crude nonaflate (405.2 g).

Step 6: The product from the previous step was dissolved in toluene (3.4 L). To this solution was added potassium carbonate (168.6 g), 3-mercapto-propionic acid ethyl ester (85.4 g), tris(dibenzylideneacetone)dipalladium(0) (Pd₂ dba₃; 2.84 g) and bis(2-diphenyl-phosphanyl)ether (DPEphos; 4.1 g). The mixture was degassed with nitrogen, before it was refluxed overnight. The mixture was cooled to 0° C., and the precipitated solid was filtered off and washed with toluene (100 mL). The combined filtrates were used in the next step.

Step 7: The product from the previous step was added to an ice-cooled suspension of potassium tert-butoxide (95.4 g) in toluene (2.8 L) over ˜2 h. Then 1-bromo-2-fluoro-4-methyl-benzene (121 g), tris(dibenzylideneacetone)dipalladium(0) (Pd₂ dba₃;1.7 g) and bis(2-diphenyl-phosphanyl)ether (DPEphos; 2.48 g) were added, and the mixture was refluxed for ˜1 h. The crude mixture was cooled to room temperature and filtered through silica gel, and concentrated in vacuo to give the crude product (240 g).

Step 8: The product from the previous step was dissolved in 33% hydrogen bromide in acetic acid (368 mL; 3 equivalents HBr) and the solution was stirred at 110° C. for ˜4 h. Then additional 33% hydrogen bromide in acetic acid (˜0.5 equivalents HBr) was added, and the mixture was stirred at 110° C. for 45 minutes before it was cooled to rt. Next morning, the solution was cooled on an ice-bath, and diethyl ether (2.25 L) was added. After 1.5 h, the precipitated solid was collected by filtration to give the desired product as the hydrobromide salt (185 g).

### Example 4

### Effect on Neuropathic Pain

There are several well validated animal models of neuropathic pain available for assess of antinociceptive potential of drugs. Among the most frequently used model are chronic constriction injury models (for example Bennett and Xie, Pain, 1988) and the capsaicin (Gilchrist et al., Pain 1996) and formalin [Neuropharm., 48, 252-263, 2005; Pain, 51, 5-17, 1992] models. To demonstrate an efficacy against neuropathic pain, compound I was tested in the formalin model of neuropathic pain. In this model, mice receive an injection of formalin (4.5%, 20 μl) into the plantar surface of the left hind paw and are afterwards placed into individual glass beakers (2 l capacity) for observation. The irritation caused by the formalin injection elicits a characteristic biphasic behavioural response, as quantified by the amount of time spent licking the injured paw. The first phase (˜0-10 minutes) represents direct chemical irritation and nociception, whereas the second (˜20-30 minutes) is thought to represent pain of neuropathic origin. The two phases are separated by a quiescent period in which behaviour returns to normal. Measuring the amount of time spent licking the injured paw in the two phases assesses the effectiveness of test compounds to reduce the neuropathic-like pain response.

TABLE 1

| | Vehicle | 0.25 mg/kg s.c | 1 mg/kg s.c | 2.5 mg/kg s.c |
|---|---|---|---|---|
| 0-5 minutes | 51 | 40 | 40 | 31 |
| (sec) | | | | |
| 20-30 minutes | 66 | 48 | 21 | 7 |
| (sec) | | | | |

The data in table 1 shows that compound I has little effect in the first phase representing direct chemical irritation and nociception. More notably, the data also show a clear and dose-dependent decrease in the time spent licking the injured paw in the second phase indicating an effect of the compound of the present invention in the treatment of neuropathic pain.

1. A compound having the structure: 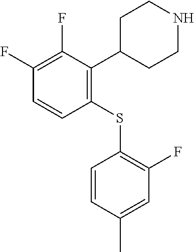
and pharmaceutically acceptable salts thereof.

2. (canceled)

3. A pharmaceutical composition comprising the compound of claim 1 and at least one pharmaceutically acceptable carrier or diluent.

4. A method of treatment of a disease selected from major depressive disorder; dysthymic disorder; mood disorder due to a general medical condition; atypical depression; seasonal affective disorder; melancholia; treatment resistant depression; depression associated with partial responders; depression associated with bipolar disorder, pain, Alzheimer's disease, psychosis, Parkinson's disease, Lewy body disease, Huntington's disease, multiple sclerosis or anxiety; general anxiety disorder; social anxiety disorder; panic attacks; phobia; social phobia, obsessive compulsive disorder; post traumatic stress disorder; acute stress; Attention Deficit Hyperactivity Disorder (ADHD); and pain, comprising the administration of a therapeutically effective amount of the compound of claim 1 to a patient in need thereof.

5-6. (canceled)
